(19) United States
(12) Patent Application Publication
(10) Pub. No.: US 2022/0160581 A1
(43) Pub. Date: May 26, 2022

(54) TESTING KIT

(71) Applicant: Respcair Medical Ltd, Dublin (IE)

(72) Inventor: Oran Rigby, Dublin (IE)

(21) Appl. No.: 17/534,231

(22) Filed: Nov. 23, 2021

(30) Foreign Application Priority Data
Nov. 25, 2020 (GB) 2018511.2

(51) Int. Cl. A61J 1/16; H04B 1/3877

(52) U.S. Cl. CPC A61J 1/16; H04B 1/3877

(57) ABSTRACT

A base assembly for a testing kit comprises: a smart phone holder; a vial holder; and a vial storage area. The base has: a depth sufficient that when a smart phone including a camera is disposed within the smart phone holder, the smart phone is far enough away from a vial held in the vial holder that at least the vial can be imaged by the camera at a known distance and location relative to the camera. The base has a width sufficient that the holder can accommodate the smart phone and that the base can store a vial laid flat on the base during transport. The base has a height sufficient that the smart phone holder can maintain the smart phone upright within the holder, that the vial holder can maintain the vial upright within the vial holder during sample collection and that the base can store a vial laid flat on the base during transport.

### RELATED APPLICATIONS

This application claims priority to United Kingdom patent application no. 2018511.2, filed Nov. 25, 2020, the disclosure of which is hereby incorporated herein by reference in its entirety.

### FIELD OF THE DISCLOSURE

The present invention relates to a base assembly for a testing kit.

### BACKGROUND

In recent years, at home DNA testing kits have gained significant popularity. They offer individuals the opportunity to gain insight into their health, origins, genetics and traits from the comfort of their homes. The two most prominent methods of sample collection for users are buccal swabs and saliva collection as they have been shown to have comparable DNA yields to blood samples. Saliva collection is the most commonly used method for DNA collection in commercially available DNA testing kits.

Procedurally, these at home DNA collection kits offer simple, easy to follow instructions for users to collect their biological specimen. Saliva collection typically starts with informing users not to eat or drink 30 minutes prior to the collection of their saliva sample. Users are then instructed to spit 2 milliliters—up to a marked line—of saliva into a vial via a funnel. Depending on the kit there are slight variations upon how to secure the sample but in general a stabilizing solution is released into the specimen and a cap is secured onto the vial.

There are two main saliva collection vials:

The vial in the DNA kit provided by 23andme™, comes with two caps. The user secures a funnel lid prior to saliva collection and after collection closes the lid to release a stabilizing liquid. The user then unscrews the funnel lid completely and screws on a tube cap.

The vial from Spectrum Solutions™ comes with a funnel attached, whereby the user deposits their saliva via the funnel, and then unscrews the funnel and secures a second cap which releases the stabilizing liquid when screwed in place.

In both cases, the user then places their vial in a biohazard bag and ships the vial to the company's lab for the DNA test.

Testing for infections such as COVID-19 is rapidly evolving and while standards do exist, new studies are continually emerging which challenge the established view of the most appropriate, effective and manageable methods for testing. At-home testing offers unique benefits. Not only is it convenient for users, but it limits health care staff's exposure to potential cases and reduces crowding at testing facilities. It also offers a way to vet employees or students prior to their return to work or campuses. The potential for diagnosis, treatment and recovery to all take place within one's home not only promotes social distancing, which aids in minimizing the spread, but also frees up hospitals and clinics to provide care for the more severe cases.

There are some significant drawbacks of at-home testing which must be addressed. For example, unsupervised testing can lead users to doubt themselves or simply perform the test inaccurately.

Thus, there is a need for an at-home collection kit to be intuitive and to instill confidence in the user that they are performing the test correctly.

### SUMMARY

According to the present invention, there is provided a base assembly for a testing kit according to claim 1.

Preferably, the base assembly includes a location to place a smart phone, a location to secure an instruction card, a vial and a removable vial holder.

Embodiments enable a repeatable, effective and user friendly at-home testing kit for collection of biological samples for DNA, drug and diagnostic testing including but not limited to infections such as COVID-19, without direct clinical supervision.

Testing kits including the base assembly can also include an alcohol wipe and biohazard bag. There may also be additional items depending on the type of test, for example, drug, steroid, sexually transmitted infections (STIs).

The instruction card can show graphically the procedure the user should follow.

In some cases, the user can also be guided by an application running on the smartphone, but it can be sufficient for the phone simply to acquire a photograph or video of the user conducting the test with both their face and the sample collection device in view to authenticate the test.

Kits including the base assembly of the present invention come prepared and suitable for integration with an application which is easily installed and executed on a variety of smart phones and assists with conducting a collection procedure intuitively and repeatably.

Embodiments provide the ability to positively identify the individual who is providing a sample for collection to ensure that the genetic profiling and associated testing, sequencing and results are correctly attributed to the specific individual.

This is vital not only for ensuring individuals who partake in clinical trials using their genomic data are the clinically appropriate person but also for reasons of public health tracing and a host of privacy and health implications.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

An embodiment of the invention will now be described, by way of example, with reference to the accompanying drawings, in which:

FIG. 1 is a perspective view of base assembly for a testing kit according to an embodiment of the present invention;

FIG. 2 is a top view of the base assembly of FIG. 1;

FIG. 3 is a front view of the base assembly of FIG. 1;

FIG. 4 is a right view of the base assembly of FIG. 1;

FIG. 5 is an exploded perspective view of the base assembly of FIG. 1;

FIG. 6 is a perspective view of a vial holder component for the base assembly of FIG. 1;

FIG. 7 shows an exemplary vial for use with the base assembly of FIG. 1;

FIG. 8 shows the funnel in place on the vial;

FIG. 9 shows the cap before being fitted on the vial; and

FIGS. 10 and 11 illustrate a base assembly according to an alternative embodiment of the present invention.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

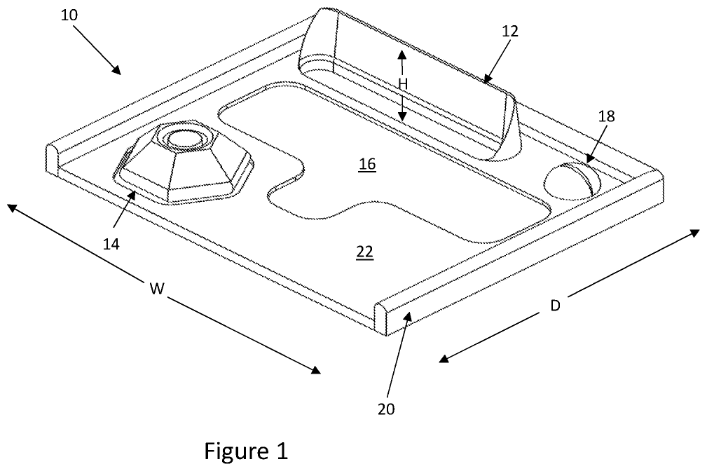

Referring now to the drawings, FIG. 1 shows an exemplary base assembly 10 for a testing kit according to an embodiment of the present invention.

The base is generally rectangular and in the embodiment comprises 5 main features defined in the base:

a smart phone holder 12;

a vial holder 14;

a vial storage area 16;

an instruction card holder 18; and

an area 22 where a unique code, such as a QR code, identifying the test kit can be displayed.

The base can be fabricated from any number of different types of material and using any number of fabrication techniques including folding card or injection moulding plastics materials. The base can also be formed by molding pulp material.

For an understanding of the scale of the assembly, exemplary dimensions for the overall width (W), depth (D) and height (H) of the base are approximately 170 mm×140 mm×30 mm. The critical dimensions are that the base needs to be deep enough (D) so that when a smart phone (not shown) is disposed within the holder 12, it is far enough away from a vial held in the vial holder 14 that the vial can be imaged by the phone camera and preferably that a user conducting the test and sitting in front of the testing kit, with the vial between the user and the phone can also be imaged—either to identify and/or authenticate the user performing the test and/or to monitor the user as they perform the test. The base 10 needs to be wide enough (W) that the holder 12 can in turn be wide enough to accommodate a variety of smart phones and also that the base 10 can store a vial laid flat on the base during transport. The base also needs to be high enough (H) to allow the holder 12 to maintain a smart phone upright within the holder 12 and to allow the vial holder 14 to maintain a vial upright within the holder 12 during the test. The base 10 including the various other contents required to perform the test can be slid inside a closely conforming sleeve for transport, but even without such a sleeve, the phone holder 12 and vial holder 14 can project high enough from the base that they protect a vial laid flat between the two holders 12, 14 during transport.

In the embodiment, a peripheral wall 20 with a height lower than the height of the phone holder, runs around three sides of the base, other than the front of the base. Such a wall can help provide the required robustness for the kit during transport and when being employed during the test as well as help to maintain the contents of the kit on the base when it is removed from a sleeve or package.

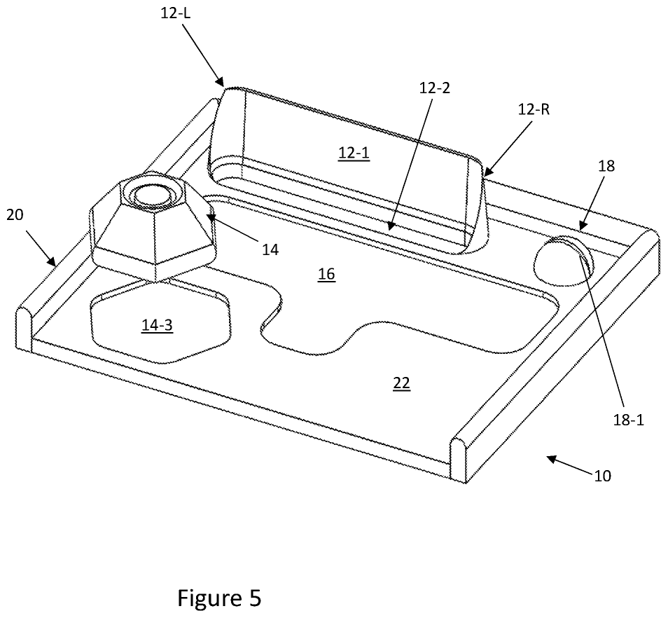

Turning now to the components in more detail, as shown in FIG. 5 the phone holder 12 comprises a central wall 12-1 running parallel to the back of the base and projecting upwards from the base at a slight angle inclined towards the back of the base. An aperture 12-2 is defined in the base and runs along the central wall 12-1, so that a phone can locate in the aperture 12-2 and lean against the wall 12-1 with a phone camera facing towards the front of the base. (In variants of the embodiment, the aperture 12-2 need not pass completely through the base and may comprise a recess.) The aperture 12-2 is approximately 15 mm deep (in the direction D) and approximately 110 mm long (in the direction W) with circular rounded ends—as this is sufficient to accommodate most phones, especially when they are carried in a case. The ends 12-L, 12-R of the upstanding central wall 12-1 curve around the rounded ends of the aperture 12-2 so as to locate a smartphone laterally within the base 10 with the field of view of the camera extending laterally around the location of the vial holder 14.

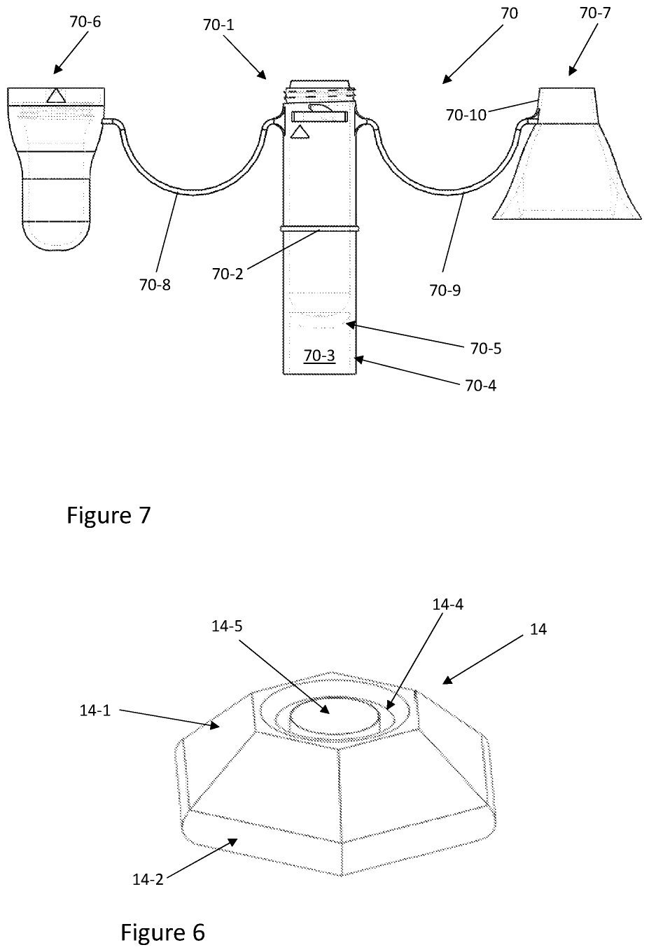

Referring now to FIG. 6, the vial holder 14 is shown in more detail. In the embodiment, the holder comprises a frusto-hexagonal body outer surface 14-1 with a depending skirt 14-2. The vial holder 14, and the skirt 14-2 especially, is sized so that it can push fit into a corresponding aperture 14-3 in the base, FIG. 1. (In variants of the embodiment, the aperture 14-3 need not pass completely through the base and may comprise a recess.) The outline of the aperture 14-3 need not conform precisely with the outline of the skirt 14-2 of the vial holder 14, but it should be shaped so that it grips the vial holder 14 and securely retains the vial holder 14 in the base.

Fabricating a vial holder 14 separate from the base 10 facilitates providing a vial with active electrical and/or electronic elements (not shown). So for example, the vial holder 14 could incorporate a heating element which when actuated causes a saliva sample held in the vial to react with a stabilising solution and in some cases to change colour to indicate that the test has been completed correctly and/or to indicate the outcome of the test.

The Center for Disease Control and Prevention (CDC) recommends as a stabilising solution Hanks Balanced Salt Solution with calcium and magnesium ions. Phenol red can be added as a pH indicator supplemented with heat activated fetal bovine serum, gentamicin sulphate (or equivalent antibiotic), and amphotericin B (or equivalent antifungal). The phenol red pH indicator in the Hanks salt solution colours the solution red, ideal colour indicator for detection with an application running on a smart phone located in the phone holder 12 during sample collection.

On the other hand, if heating of the sample is not required, then the vial holder 14 can be integrally formed with the rest of the base 14.

In any case, in the embodiment, a cylindrical depression 14-4 is formed in the top surface of the vial holder 14. The depression is suitable for receiving a cylindrical vial 70 described more detail in relation to FIG. 7.

In the present embodiment, the vial 70 used with the base assembly comprises three main bodies. A main saliva collection vial 70-1 can hold approximately 5.5 millilitres—which is sufficient for saliva collection and any bubbles formed, as well the stabilising solution when dispensed into the saliva collection vial 70-1. A protruding ring 70-2 extends around the vial located at a height corresponding to a volume of 2 ml within the collection portion of the vial to aid the user in knowing they have collected enough saliva. (Clearly, any number of different indicia can be employed to indicate any desired volume within the vial 70-1.)

The bottom portion of the vial 70-1 is hollow 70-3 and defines a downwardly depending skirt 70-4 which fits into the depression 14-4 formed in the vial holder 14 with a bottom surface 70-5 of the saliva collection chamber bearing against the remaining upper surface 14-5 of the vial holder surrounded by the depression 14-4. This ideally locates the vial 70-1 against the vial holder 14 in cases where the vial holder includes a heating element allowing the collected sample within the vial to react with stabilizing solution which has been dispensed into the vial.

Another benefit to the vial holder 14 being of relatively larger diameter than the vial 70 is that it allows for the user to hold something larger than the vial 70, aiding in a more secure grip without blocking the view of the vial 70-1 from a smart phone camera located in the phone holder 12. Indeed in variants of the illustrated embodiment, the vial holder 14 could be more ergonomically shaped or be provided with indicia to encourage users to grip the vial holder 14 in a certain way to ensure the view from the camera is not obscured when a sample is being provided.

In any case, it will be seen that the saliva collection vial 70-1 protrudes from the vial holder 14 to a sufficient height that as well as the field of view of the camera extending laterally around the location of the vial holder 14, the field of view of the camera also extends vertically along and above the length of the vial 70.

The saliva collection vial 70-1 comes with a cap 70-6 and a funnel 70-7 attached through respective leads 70-8, 70-9 extending diametrically away from each side of the vial 70-1 and during use each can be broken off at the appropriate step of the collection protocol as described below. As will be seen from FIG. 7, the saliva collection vial 70-1 is substantially longer than each of the cap 70-6 and the funnel 70-7 and the form a T-shape generally conforming to the shape of the vial storage area 16 defined in the base 10.

A suitable material for the vial 70 is polypropylene as this is sufficiently flexible for the bends in the shape of the vial and also sufficiently clear to aid in visibility for specimen collection. Additionally, membrane material maintaining the stabilizing solution within the cap 70-6 needs to be non-reactive with the solution and have no effect on the sample viability during transport after it has been perforated—one possible solution for this is parafilm.

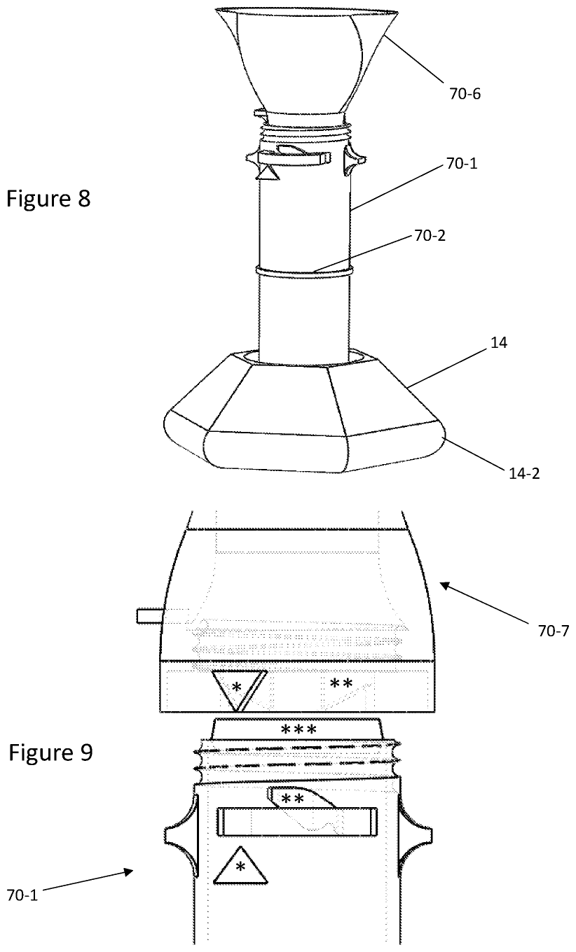

When providing a sample, a user breaks off the funnel 70-7 and inserts it into the top of the saliva collection vial 70-1, FIG. 8. The funnel 70-7 has a long-tapered end 70-10 to allow for a secure fit into the top of the saliva collection vial 70-1 without requiring any twisting. It will nonetheless be appreciated that alternative mechanisms than a push fit can also be employed. After the full volume of saliva has been collected from the user, evidenced by the 2 ml line 70-2, the funnel 70-7 can simply be removed from the vial 70-1 and discarded by the user.

After complete specimen collection, the cap 70-6 is broken off. FIG. 9 shows in detail a bottom portion of the cap 70-6 being offered to the top of the saliva collection vial 70-1 just before engagement. First, when the cap 70-6 is screwed onto the vial 70-1, a lip (indicated by three asterisks ***) perforates a frangible membrane which holds the stabilising solution in the cap 70-6. This releases the solution into the vial 70-1. Two pairs of asterisks ** indicate a breakable tab on the saliva collection vial 70-1 and an angled protrusion on the cap 70-7. When twisting on the cap 70-6, the protrusions push the tab down slightly, just enough to continue screwing on. However, if the cap is unscrewed, the angle of the protrusion will lift the tab up and consequently break it off. This mechanism allows for an obvious way to see if a vial has been tampered with when it is received at the lab. An intact tab indicates that the cap was never unscrewed, while no tab indicates the cap was unscrewed at some point.

When the cap 70-6 is securely closed, the arrows on each of the cap 70-6 and vial 70-1, indicated by a pair of single * asterisks, will align together. From this closed position the tab is not accessible to be tampered with and the specimen is securely stored in the vial.

The instruction card holder 18 is formed towards the back of the base 10 adjacent to the phone holder 12. The holder 18 has a slot 18-1 defined therein running parallel to the phone holder 12 so that an instruction card (not shown) provided with the kit can be fitted into the slot with the instructions facing a user providing the sample, so that the user can both view the instruction card and remain within the field of view of the camera while providing the sample. The instruction card holder 18 may not be required in cases where sample collection is driven by an application running on the phone, but it may nonetheless be provided to supplement the application running on the phone or as an alternative if the user is not running an application at the time of sample collection. In that case, the phone may simply be recording the user with the recording being provided for later analysis/verification of the collected sample.

Finally, the base incorporates an area 22 adjacent the front of the base where a unique code, such as a QR code, identifying the test kit can be displayed. The code (not shown) can be printed directly on the surface of the base in the area 22; or the code can be first printed on a label, possibly an adhesive label, and affixed to or otherwise located on the base within the area 22. A corresponding code can also be provided on the vial 70, although it will be appreciated that any such code on the vial can tend to obscure the vial or the contents of the vial.

Now that the components for kit have been described, one protocol a user may need to follow to provide a sample using the kit could comprise:
1. Do not eat, drink, smoke, chew gum, brush teeth or use mouthwash for at least 30 minutes prior to providing your sample.
2. Cough deeply 2 to 3 times, allowing phlegm and secretions to collect in mouth.
3. Collect about 2 ml of saliva in the vial, excluding bubbles.
4. Add the stabilizing solution.
5. Cap securely.
   Thus, an application running on a phone located in the holder 12 during sample collection might:
Have the user verify that they have not been eating, drinking, etc. within last 30 minutes; and possibly allow the user to proceed anyway, if desired;
Verify the user's identity by having the user enter a one-time code and date of birth and create a unique test ID with the pair;
Verify the presence of a human face within the field of view of the phone when located in the base 10 and acquire a photograph of the user for verification;
Verify the vial/test kit by reading a QR code: provided in the space 22; printed on the vial 70; and/or printed on an instruction card (before it is (re-)placed in the instruction card holder 18) included with the kit, decode and save the value into log file. In some embodiments, the user can begin the process by first imaging the QR code with their smart phone before it is placed in the phone holder. This can cause the application to be installed (if not already) and launched, so providing the possibility of the application automatically guiding the user through the process from the point of opening the test kit through to completion of the test;
Verify the user coughing deeply 3 times using audio input;
The application may also include classification software trained to identify a user collecting phlegm and secretions within their mouth and spitting into the saliva collection vial 70-1, i.e. rather than providing a sample from a source other than the user. In this regard, the software can verify that, from the time the vial 70 is first placed in the vial holder 12 until the cap 70-6 is secured to the saliva collection vial 70-1, the vial is not removed from the vial holder so that the authenticity and validity of the user providing the sampled can be verified. Locating the vial 70-1 and the user within the field of view of a smart phone located in the phone holder 12 facilitates the operation of classification software trained for this purpose, as the size of the vial 70-1 and the location of the vial 70-1 relative to the phone when located within the base 10 are known and so they make the training and operation of the software for identifying that the user has actually coughed and provided their own saliva correctly into the vial 70-1 more robust.
The application can verify that the cap 70-6 has been sealed to the vial 70-1 using a color test to detect the collected sample changing color. Again, because the vial 70-1 is of a known size and the location of the vial 70-1 relative to the phone when located within the base 10 is known—training classification software to locate the region of the field of view of the camera in which to detect the contents of the vial 70-1 changing color is relatively easy and robust. Detecting that the user has successfully placed the cap 70-6 on the vial 70-1 can also act as a trigger to stop video recording of the sample collection. Alternatively, once the application detects that the cap 70-6 is on the vial 70-1 through simple image recognition, it can wait for interaction or any signal from the user to signal that sample collection is complete before stopping recording. Once the vial 70-1 has remained secured in the vial holder 14 throughout the test from the time the user has been recognized within the field of view of the smart phone to sealing the cap 70-6 on the vial 70-1, the collected sample can be treated as valid.
Finally, the application can record and save the video of the user from the “photograph verification” step to the “verify seal using color test” steps.

Nonetheless, it will be appreciated that even without a dedicated application running on the phone, simply having the phone record the user from first presenting themselves in front of the kit with a view to providing the sample through to sealing the cap 70-6 to the vial 70-1 both encourages proper performance of the test and allows for subsequent verification of the test possibly even by automated software running at a location remote from the original phone.

Still further variations of the above described embodiment are possible, for example, rather than a rigid component, which does facilitate reliable manufacture and robust performance, one of more components of the base 10 including the phone holder 12, the instruction card holder 18 or the vial holder 14 may comprise components which fold from a stowed state for transport to a deployed state for performing the test.

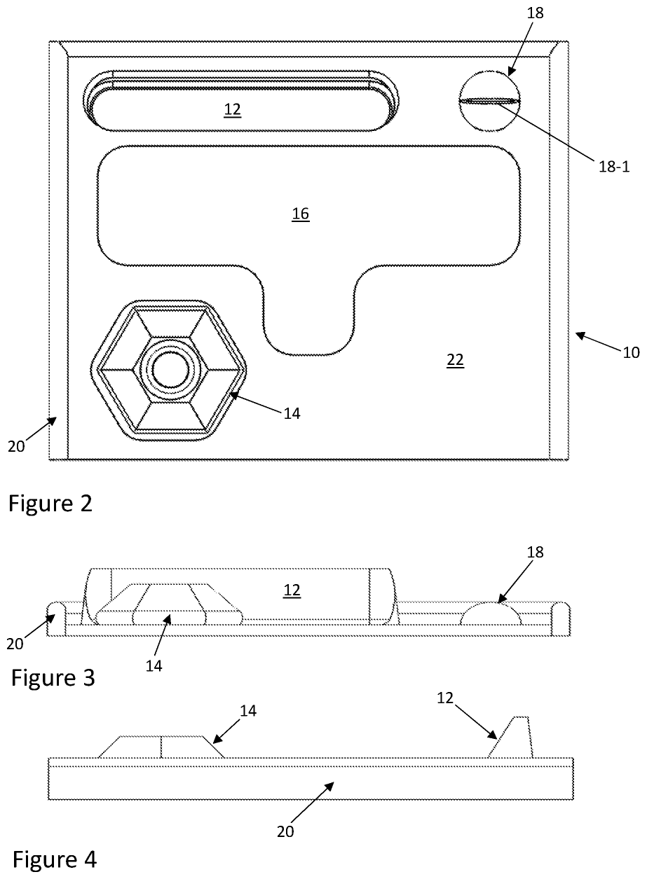

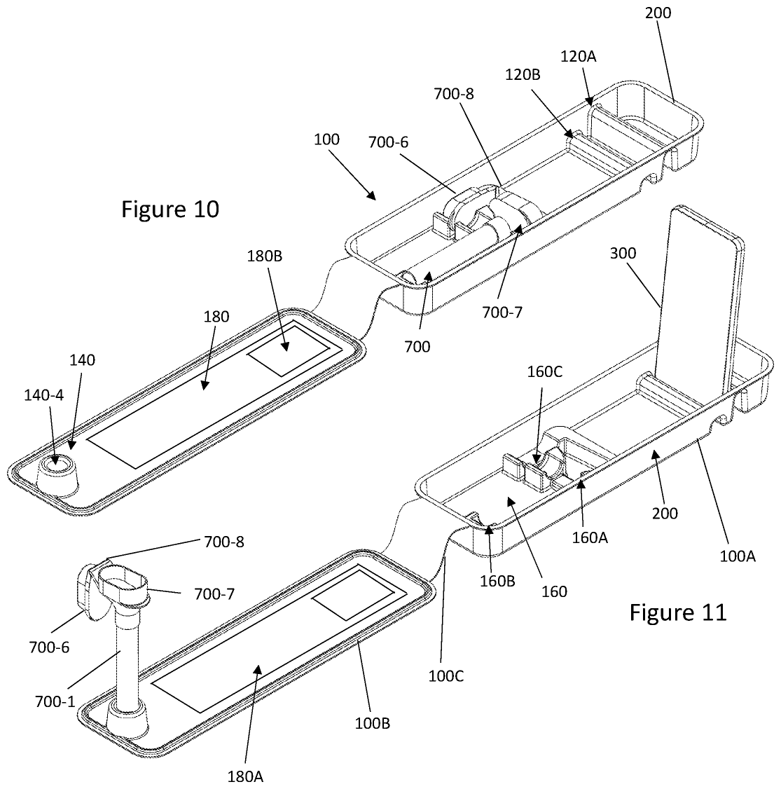

Referring now to FIGS. 10 and 11, there is shown a base assembly 100 for a testing kit according to an alternative embodiment of the present invention. In the example, similar reference numerals are used for parts corresponding to the parts of FIGS. 1-9. In the embodiment, the assembly divides into two parts 100A and 1008 connected to one another through a live hinge 100C. These parts can comprise a plastics material formed in a single shot injection molding process; or if the live hinge 100C is to be formed of a different material than the connected parts 100A, 1008, a two-shot molding process can be employed. Alternatively, the base assembly 100 can be formed from a cardboard material.

It will be understood that while a live hinge facilitates the base assembly 100 being formed from a single part, in alternatives to the illustrated embodiment, the parts 100A and 1008 could be formed separately and connected through a multi-part hinge.

The first part 100A of the base assembly is generally elongated with a peripheral wall 200 and a generally recessed floor. The live hinge 100C extends from an upper lip of a short end of the peripheral wall 200. At the end of the part 100A remote from the hinge, a phone holder is formed comprising a pair of spaced apart parallel ridges 120A, 1208 extending across the width of the part 100A. The ridge 120A further from the hinge 100C extends higher from the base than the nearer ridge 1208 and the ridges are spaced apart sufficiently that a typical smartphone 300 can be securely held between the ridges with its front facing camera directed towards the opposite end of the base assembly 100. In the example shown, the smartphone 300 sits between the ridges 120A, 120B so that it is angled upwardly away from the base, ensuring that the field of view of the camera will be suitable for capturing a user performing the test.

The second part 1008 of the base forms a lid for the assembly when folded over the first part 100A. A vial holder 140 is formed towards the end of the part 1008 remote from the hinge 100C. The vial holder 140 is located so that when the second part 1008 is folded over the first part 100A, the vial holder 140 will locate in a recess between the first ridge 120A and the wall 200 of the first part 100A. In some cases, the recess can be sized so that the vial holder fits into the recess and holds the second part 1008 closed over the first part 100A. In other embodiments, the periphery of the second part 1008 can be shaped so that it snap fits with the wall 200 of the first part 100A to hold the second part 1008 closed over the first part 100A. In other examples, an external wrapper (not shown) could be used to hold the second part 1008 closed over the first part 100A. Indeed, more than one of these techniques can be combined or another suitable closing mechanism can be employed.

The vial holder 140 comprises a generally cylindrical wall defining a cylindrical depression 140-4 large enough in diameter and high enough to hold a vial 700 while allowing the contents of the vial to be imaged by the smartphone during the performance of the test.

In the embodiment, the recess between the phone holder ridge 120B and the hinged end of the wall 200 is employed as a vial storage area 160. In the embodiment, the vial storage area 160 includes a separately formed removable insert. However, it will be appreciated that the features of the insert could equally be integrally formed within the base part 100A.

In any case, the vial storage area 160 includes a pair of spaced apart protrusions 160A, 1608 which are sized and shaped to grip and hold a vial 700, lengthways within the part 100A both before performing the test and after the test has been performed. In the example of FIGS. 10 and 11, the vial 700 comprises a funnel 700-7 integrally formed and extending from the top of a cylindrical vial body 700-1 which facilitates the user providing their sample during the test. In this case, the funnel has an oblong section being substantially the same or only marginally wider than the diameter of the cylindrical vial body 700-1 in one direction and being substantially wider than the diameter of the vial body 700-1 in a transverse direction.

It will be understood that the dimensions of the base are such that it is wide enough to accommodate the smartphone within the holder 120A, 1208, and the wall 200 is high enough to accommodate the stored vial 700 before and after performing the test. Thus, the oblong funnel shape allows the overall height of the wall 200 to be minimized, but facilitates the user providing their sample. It will be appreciated that the width of the part could be made narrower by providing gaps in the wall 200 in the space between the ridges 120A, 1208 so allowing the side of the smartphone to extend, possibly only slightly, beyond the base during the performance of a test.

In the embodiment, a live hinge 700-8 extends from an upper lip of the funnel 700-7. A cap 700-6 is formed at the remote end of the hinge. Again, the vial storage area 160 comprises a protrusion 160C shaped to receive the cap 700-6 and defines a path to accommodate the hinge 700-8 when the vial 700 is stored in the base.

Once the test is complete, the user simply needs to fold the cap 700-6 into the funnel 700-7 to seal the contents of the funnel. In this case, the seal can be provided through a push fit of the two components, but suitable interlocking features can also be defined on the cap and funnel to facilitate positive interconnection of these components. In the embodiment, the cap 700-6 largely conforms to the interior shape of the funnel 700-7 so that the external shape of this portion of the vial does not change when the cap locates in the funnel after the test, so allowing the vial 700 to be replaced within the protrusions 160A and 1608 once the test is complete.

The length of the base is such that when extended, the vial holder 140 is spaced far enough from the smartphone 300 that the vial 700 and the user are within the field of view of the camera while the test is performed.

In the embodiment, the length of the part 1008 is such that the space between the vial holder 140 and the hinged end of the part 1008 provides a suitable surface for receiving both the written instructions 180A guiding the user through the test, as well as a QR code 1808 for identifying the test. Each of these can be printed on a single adhesive label.

As will be appreciated, the test kit including the base assembly 100 and vial 700 can be shipped with the part 1008 folded over and closing the part 1008 with the vial 700 securely stored. Once a user opens the base, they immediately see the instructions 180A explaining how the test is to be performed. These may include instructions indicating to the user the application they need to obtain and execute in order to guide them through and monitor their correct performance of the test as described above. Once complete, the vial 700 can be replaced within the protrusions 160A, 1608; the smartphone 300 removed; the part 1008 closed over the part 100A and the kit returned for any further analysis which may be required of the sample provided.

As in the case of the embodiment of FIGS. 1-9, it will be appreciated that the embodiment of FIGS. 10 and 11 can be adapted, for example, to incorporate a heater within the vial holder 140.

While the embodiment of FIGS. 10 and 11 has been illustrated with the second part 1008 comprising a flat lid, it will be appreciated that in variants of the embodiment, the second part 1008 could also be recessed to provide a deeper container for storing test materials.

While the above described embodiments have been based on a test involving saliva collection, it will be appreciated that the base assembly could be used for buccal swab collection.

Those skilled in the art will recognize improvements and modifications to the preferred embodiments of the present disclosure. All such improvements and modifications are considered within the scope of the concepts disclosed herein and the claims that follow.

What is claimed is:

1. A base assembly for a testing kit comprising:
a smart phone holder;
a vial holder; and
a vial storage area
wherein the base has: a depth sufficient that when a smart phone including a camera is disposed within the smart phone holder, the smart phone is far enough away from a vial held in the vial holder that at least the vial can be imaged by the camera at a known distance and location relative to the camera; a width sufficient that the holder can accommodate said smart phone and that the base can store a vial laid flat on the base during transport; and a height sufficient that the smart phone holder can maintain said smart phone upright within the holder, that the vial holder can maintain said vial upright within the vial holder during sample collection and that the base can store a vial laid flat on the base during transport.

2. The base assembly according to claim 1 wherein the vial storage area comprises a recess formed in the base between the smart phone holder and the vial holder and conforming generally to the shape of the vial.

3. The base assembly according to claim 1 further comprising an instruction card holder configured to hold an instruction card within the field of view of a user performing the test while facing the base assembly.

4. The base assembly according to claim 1 wherein the base is formed from one or more of: pulped material; card; or plastics material.

5. The base assembly according to claim 1 wherein the vial holder is formed separately from the base and wherein the vial holder includes a heater element actuable to heat a sample collected in the vial when the vial is located in the vial holder.

6. The base assembly according to claim 1 wherein the vial holder is integrally formed with the base.

7. The base assembly according to claim 1 where the base is approximately 170 mm in width, 140 mm in depth and 30 mm high.

8. The base assembly according to claim 1 wherein the phone holder comprises a wall upstanding from the base at an incline to a plane of the base.

9. The base assembly according to claim 1 wherein the base further comprises an area where a unique code identifying the test kit is displayed.

10. The base assembly according to claim 9 where the unique code comprises a QR code.

11. The base assembly according to claim 1 wherein the base comprises two parts connected to one another through a hinge located between said smart phone holder and said vial holder.

12. The base assembly according to claim 1 wherein a first part of said base is recessed and configured to include said smart phone holder and said vial storage area; and a second part of said base is configured to include said vial holder, said hinge enabling said second part to close over said first part with a vial stored therein.

13. The base assembly according to claim 12 wherein said second part is either: generally flat or recessed.

14. A kit comprising a base assembly, the base assembly comprising:
a smart phone holder;
a vial holder; and
a vial storage area; wherein:
the base has: a depth sufficient that when a smart phone including a camera is disposed within the smart phone holder, the smart phone is far enough away from a vial held in the vial holder that at least the vial can be imaged by the camera at a known distance and location relative to the camera; a width sufficient that the holder can accommodate said smart phone and that the base can store a vial laid flat on the base during transport; and a height sufficient that the smart phone holder can maintain said smart phone upright within the holder, that the vial holder can maintain said vial upright within the vial holder during sample collection and that the base can store a vial laid flat on the base during transport; and
the kit further comprises a vial comprising:
a saliva collection vial;
a funnel connectable to the saliva collection vial and configured to funnel saliva into the saliva collection vial; and
a cap configured to seal said saliva collection vial once said funnel has been removed.

15. The kit according to claim 14 wherein said cap comprises a chamber with a frangible membrane and storing a stabilizing solution, said membrane being broken when said cap seals said saliva collection vial.

16. The kit of claim 14 wherein said saliva collection vial, funnel and cap are integrally formed.

17. The kit of claim 14 further comprising an instruction card and a bio-hazard bag.

18. A method for performing a test, the method comprising:
providing a kit, the kit comprising a base assembly, the base assembly comprising:
a smart phone holder;
a vial holder; and
a vial storage area; wherein:
the base has: a depth sufficient that when a smart phone including a camera is disposed within the smart phone holder, the smart phone is far enough away from a vial held in the vial holder that at least the vial can be imaged by the camera at a known distance and location relative to the camera; a width sufficient that the holder can accommodate said smart phone and that the base can store a vial laid flat on the base during transport; and a height sufficient that the smart phone holder can maintain said smart phone upright within the holder, that the vial holder can maintain said vial upright within the vial holder during sample collection and that the base can store a vial laid flat on the base during transport; and
the kit further comprises a vial comprising:
a saliva collection vial;
a funnel connectable to the saliva collection vial and configured to funnel saliva into the saliva collection vial; and
a cap configured to seal said saliva collection vial once said funnel has been removed; wherein the method further comprises:
receiving the vial in the vial holder;
receiving a smartphone including a camera and executing a test application configured to monitor a user performing said test in said smartphone holder, said vial and said user being within the field of view of said camera during the performance of said test;
receiving in said vial, a test sample from said user through said funnel;
sealing said vial including said test sample with said cap; and
storing said sealed vial within the vial storage area.

19. The method according to claim 18 where the test application is configured to perform one or more of the following steps:
have the user verify that they have not been eating or drinking within a given period before performing the test;
verify the user's identity;
verify the presence of a human face within the field of view of the phone when located in the base and acquire a photograph of the user;
verify the kit by acquiring an image of a code associated with the kit;
using an audio input for said smartphone, verify the user coughing deeply one or more times;
identify the user collecting phlegm and secretions within their mouth and spitting into the vial;
verify that, from a time the vial is first placed in the vial holder until the cap is secured to the saliva collection vial, the vial is not removed from the vial holder;
verify that the cap has been sealed to the vial; and
record and save a video of the user performing the test.
